(19) United States
(12) Patent Application Publication
(10) Pub. No.: US 2004/0225246 A1
(43) Pub. Date: Nov. 11, 2004

(54) Splint and treatment method for toe injuries

(76) Inventor: David G. Doctor, Amherst, MA (US)

Correspondence Address: OSTROLENK FABER GERB & SOFFEN, 1180 AVENUE OF THE AMERICAS, NEW YORK, NY, 100368403

(21) Appl. No.: 10/838,970

(22) Filed: May 4, 2004

Related U.S. Application Data
(60) Provisional application No. 60/468,210, filed on May 6, 2003.

(51) Int. Cl. A61F 5/00

(52) U.S. Cl. 602/30

(57) ABSTRACT

A splint and a method for treating toe injuries. A sleeve is sized for splinting an injured toe; a strap on the sleeve extends from the sleeve, around an adjacent toe and back to the sleeve; and fastening arrangements associated with the sleeve and the strap for fastening both ends of the strap to the sleeve, whereby the strap securely holds the splinted toe to the adjacent toe. The adjacent anchoring toe is preferably disposed toward the middle of the body from the injured toe. The sleeve advantageously applies compression to the injured toe. Preferably, one end of the strap is permanently attached to a distal end of the sleeve and is extended around a distal portion of the adjacent anchoring toe. The fastening arrangements may comprise a hook-and-loop-type fastener.

### CROSS-REFERENCE TO RELATED APPLICATIONS

This application is based upon and claims priority of U.S. Provisional Patent Application Ser. No. 60/468,210 filed by David G. Doctor on May 6, 2003 (PP/1711-5), incorporated by reference.

### BACKGROUND OF THE INVENTION

1. Field of the Invention

The present invention relates to a toe splint comprising a sleeve and a hook-and-loop fastener, particularly for treatment of lesser toe fractures, and a treatment method using the splint.

2. Background Art

FIG. 1 shows the anatomy of the foot. The “lesser toes” are defined as all toes excluding the big toe. As shown, the toes are numbered 1-5 beginning with the big toe, the lesser toes being numbered 2-5. The side of the foot designated “medial” is the outside of toe 1 or the big toe, while the side designated “lateral” is the outside of toe 5 or the little toe.

Each lesser toe has three “phalanges” identical in nomenclature to those of the fingers, namely the proximal, middle and distal phalanges. The present invention is directed toward treatment of an injury, such as a fracture or ligament injury in any part of a lesser toe, and is particularly advantageous for treating a fracture of the proximal phalanx.

The currently preferred treatment for such injuries is buddy taping of the injured toe to the medial uninjured toe, which is considered useful for pain relief and perhaps more importantly useful for stabilizing toe fractures. Buddy taping of an injured toe 3 to an uninjured toe 2 with tape 10 is illustrated in FIG. 2.

Problems with this technique include:

a. Cumbersome and time-consuming.

b. Must continually re-apply.

c. Can harm the skin around the toe. The tape can cause skin breakdown because a fractured toe often has severely contused, reddened skin, rendering it sensitive to adhesive, especially with repeated applications.

d. Not the best option for unstable fractures or fractures which require reduction.

### SUMMARY OF THE INVENTION

It is therefore desirable to provide an improved treatment for injuries of the lesser toes.

To this end, the invention provides a splint for treating toe injuries, comprising a sleeve sized for splinting an injured toe; a strap on the sleeve having two ends and being sized for extending from the sleeve, around an adjacent toe and back to the sleeve; and fastening arrangements associated with the sleeve and the strap for fastening both ends of the strap to the sleeve, whereby the strap securely holds the splinted toe to the adjacent toe.

The invention also provides a method of treating toe injuries, comprising the steps of placing a sleeve on an injured toe; extending a strap from the sleeve, around an adjacent toe and back to the sleeve; and fastening both ends of the strap to the sleeve, whereby the strap securely holds the splinted toe to the adjacent toe.

According to a preferred technique, the adjacent anchoring toe is disposed in the medial direction (toward the middle of the body) from the injured toe.

The sleeve advantageously applies compression to the injured toe. Preferably, one end of the strap is permanently attached to a distal end of the sleeve and is extended around a distal portion of the adjacent anchoring toe, and then returned back to the injured toe and fastened to the sleeve. The fastening arrangements may comprise a hook-and-loop-type fastener.

Other features and advantages of the present invention will become apparent from the following description of embodiments of the invention which refers to the accompanying drawings.

### BRIEF DESCRIPTION OF THE DRAWINGS

FIG. 1 shows the anatomy of the foot.

FIG. 2 illustrates the technique of buddy-taping an injured toe to an adjacent toe.

FIG. 3A is a plan view of a first embodiment of the invention.

FIGS. 3B and 3C are respectively a plan view and a perspective view of a second embodiment of the invention.

FIG. 4A illustrates an injured toe, and FIG. 4B illustrates application of a splint according to an embodiment of the invention.

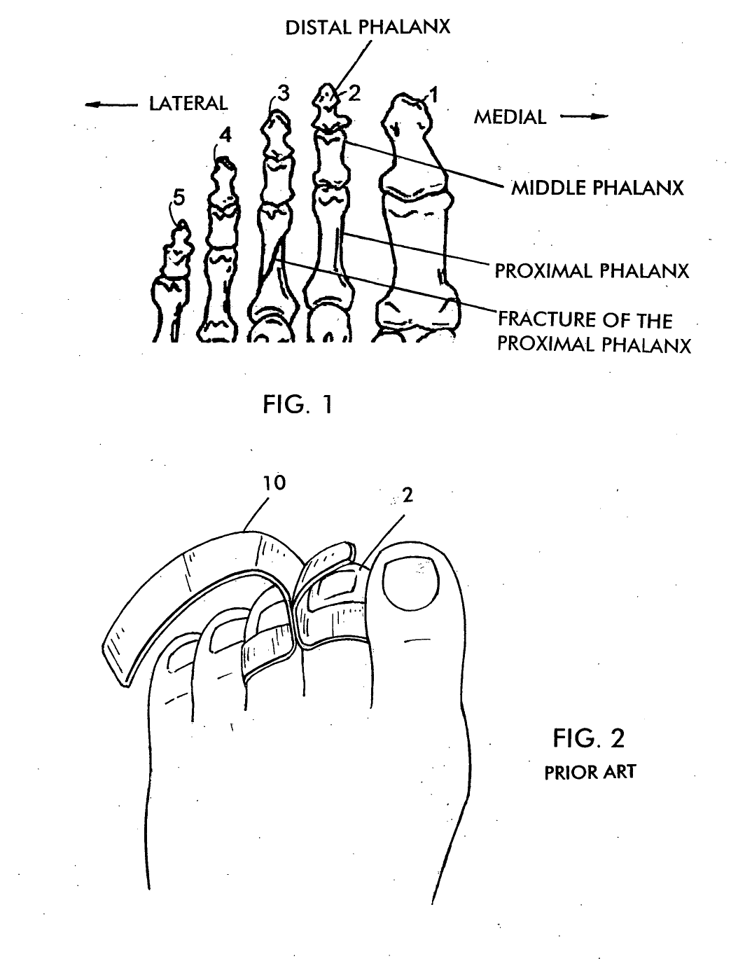

### DETAILED DESCRIPTION OF EMBODIMENTS OF THE INVENTION

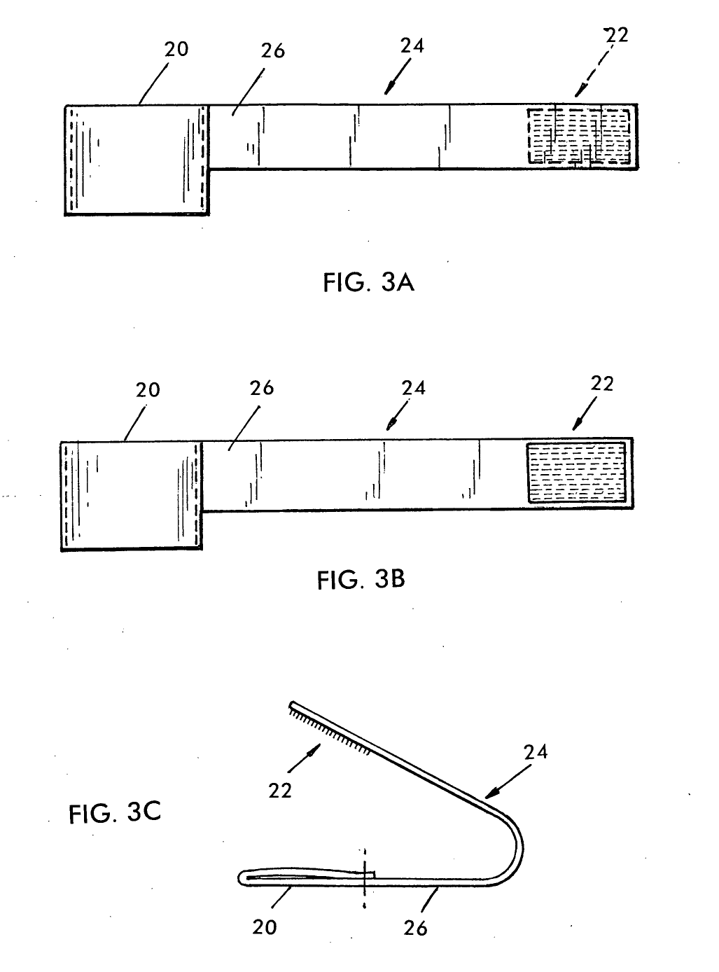

FIGS. 3A and 3B are plan views showing respective embodiments of the invention. FIG. 3C is a side view of the embodiment of FIG. 3B.

Each embodiment has a sleeve 20 of 1/16″ neoprene covered on the inside with Lycra and on the outside by UBL (unbroken-loop) material for receiving a hooked portion 22 formed on a strap 24. The base of the strap 26 is either an extension of the sleeve 20 or is formed separately and attached to the sleeve 20 by sewing or the like. The sleeve 20 is itself advantageous because it can itself stabilize the injured toe, while applying gentle uniform compression to reduce swelling in the toe. The strap 24 can be wrapped around the next toe toward the medial side and the hooked portion 22 on the strap 24 is then brought back and adhered to the UBL material on the sleeve 20.

In the embodiment of FIG. 3A, the hook portion 22 faces down (as seen in the figure) so that the strap can be passed over the top of the next toe, then around the bottom of the next toe, then back to the injured toe where the hook portion of the strap is secured to the sleeve by engagement with the UBL portion of the sleeve.

In the embodiment of FIGS. 3B and 3C, the hook portion 22 faces up, so that in use the strap is passed first around the bottom of the next toe.

One of these two embodiments may be preferred by a given patient or medical professional for reasons of comfort or ease of application, for example.

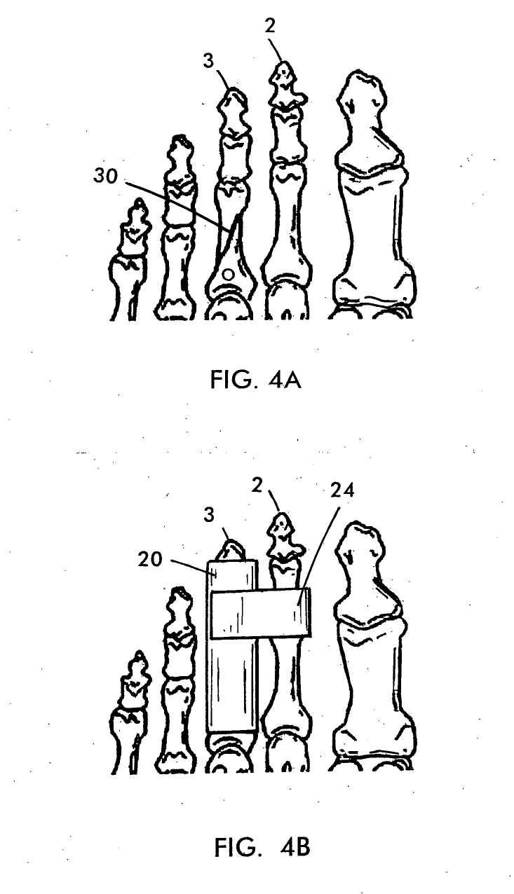

An example of the application of this device to a fracture 30 of the third proximal phalanx of an injured toe 3 is illustrated in FIGS. 4A and 4B.

As shown, it is preferable to place the strap 24 at or near the distal (tip) end of the anchoring toe 2, which achieves maximum stability. This is because the distal fracture fragment, i.e., the part of the toe beyond the fracture, is the part that has the greater tendency to change position if not adequately immobilized.

Different sleeve sizes are advantageously provided to fit injured toes of any size. It should fit snugly, but not uncomfortably. The junction of the strap 24 and the sleeve 20 can be used as a handle to gently guide the sleeve onto the injured toe with only mild pressure and therefore with minimal pain.

The device accomplishes the following:

a. replaces the cumbersome taping methods with a sleeve and a hook-and-loop fastener that does not damage the skin, is easy to apply, and is washable and reusable.

b. provides a means of reducing toe swelling, which taping cannot do.

c. provides superior stability of even the most unstable fractures of the proximal phalanx by use of a distally placed hook-and-loop anchor strap.

Although the present invention has been described in relation to particular embodiments thereof, many other variations and modifications and other uses will become apparent to those skilled in the art. Therefore, the present invention is not limited by the specific disclosure herein.

What is claimed is:

1. A splint for treating toe injuries, comprising:
a sleeve sized for splinting an injured toe;
a strap on the sleeve having two ends and being sized for extending from said sleeve, around an adjacent toe and back to said sleeve; and
fastening arrangements associated with said sleeve and said strap for fastening both ends of said strap to said sleeve, whereby said strap securely holds said splinted toe to said adjacent toe.

2. A splint as in claim 1, wherein said fastening arrangements comprise a hook-and-loop-type fastener.

3. A splint as in claim 2, wherein one end of said strap is permanently attached to said sleeve.

4. A splint as in claim 2, wherein said sleeve has loop material on an outside thereof, and said strap has hook material arranged for contacting said loop material.

5. A splint as in claim 4, wherein said hook material is disposed on a top surface of said strap.

6. A splint as in claim 4, wherein said hook material is disposed on a bottom surface of said strap.

7. A splint as in claim 2, wherein each of said sleeve and said strap has a respective one of either hook material or loop material thereon, for forming said hook-and-loop-type fastener.

8. A splint as in claim 1, wherein one end of said strap is permanently attached to a distal end of said sleeve for being extended around a distal portion of said adjacent toe.

9. A splint as in claim 8, wherein said adjacent toe is disposed in the medial direction from said injured toe.

10. A splint as in claim 1, wherein said adjacent toe is disposed in the medial direction from said injured toe.

11. A splint as in claim 1, wherein said sleeve comprises neoprene and has an inner layer of Lycra for contacting said injured toe.

12. A splint as in claim 1, wherein said sleeve applies compression to said injured toe.

13. A splint as in claim 12, wherein one end of said strap is permanently attached to a distal end of said sleeve for being extended around a distal portion of said adjacent toe.

14. A splint as in claim 13, wherein said adjacent toe is disposed in the medial direction from said injured toe.

15. A method of treating toe injuries, comprising the steps of:
placing a sleeve on an injured toe;
extending a strap from the sleeve, around an adjacent toe and back to said sleeve; and
fastening both ends of said strap to said sleeve, whereby said strap securely holds said splinted toe to said adjacent toe.

16. The method of claim 15, comprising the steps of permanently attaching one end of said strap to a distal end of said sleeve, and
extending said strap around a distal portion of said adjacent toe.

17. The method of claim 16, wherein said adjacent toe is disposed in the medial direction from said injured toe.

18. The method of claim 15, wherein said sleeve applies compression to said injured toe.
